(19) United States
(12) Patent Application Publication
(10) Pub. No.: US 2019/0090799 A1
(43) Pub. Date: Mar. 28, 2019

(54) POLYMER ALLOY COMPOSITIONS AND APPLICATION TO MEDICAL SYSTEMS AND METHODS

(71) Applicant: SABIC Global Technologies B.V., Bergen op Zoom (NL)

(72) Inventors: Yuzhen XU, Shanghai (CN); Hongtao SHI, Shanghai (CN); Shreyas CHAKRAVARTI, Evansville, IN (US)

(73) Assignee: SABIC Global Technologies B.V., Bergen op Zoom (NL)

(21) Appl. No.: 15/772,553

(22) PCT Filed: Nov. 2, 2015
(86) PCT No.: PCT/CN2015/093616

§ 371 Date: May 1, 2018

(51) Int. Cl. A61B 5/15; C08L 67/02; C08L 69/00; G01N 33/66

(52) U.S. Cl. CPC A61B 5/150358; G01N 33/66; C08L 69/00; C08L 67/02

(57) ABSTRACT

Provided herein are test elements useful in medical applications, such as glucose testing strips. The test elements have improved mechanical properties and improved processing characteristics as compared to existing test elements.

### TECHNICAL FIELD

The present application relates to the field of polymeric films used in medical applications.

### BACKGROUND

The number of people who suffer from diabetes is growing, with the Centers for Disease Control and Prevention reporting that 23.6 million Americans had diabetes in 2007, with more cases every year. With an overburdened and expensive health care system, cost-effective home diagnostics become increasingly important.

Blood glucose meters (or BGMs) are now available in pharmacies everywhere. Blood glucose measurements in such meters normally begin with a test strip. Each batch of test strips may include a code chip that contains information on the batch including the important expiration date. The code chip is inserted into the BGM; if the strips are acceptable, the test can continue. Blood Glucose Test strips are part of a complete blood glucose monitoring system. Blood is applied to the test strip and the meter is able to detect the amount of blood glucose present in the sample blood.

PET films are a traditional substrate for glucose monitoring strips. A PET film may be surface treated, followed by application of white ink to make a white PET film. PET, however, has certain mechanical characteristics (e.g., brittleness) that make it unsuitable for test strips as such strips become thinner and as the strips have new geometries and features. Because reliable function of strips and monitors is crucial, strips that perform reliably under current testing conditions and with current detector devices are important. Accordingly, there is a need in the art for improved substrate materials for blood glucose monitoring strips. The value of such materials would be enhanced if the materials were suited to continuous manufacturing processes, e.g., roll-to-roll processes.

### SUMMARY

The present disclosure provides, inter alia, compositions suitable for glucose strips and other medical applications.

### BRIEF DESCRIPTION OF THE DRAWINGS

The summary, as well as the following detailed description, is further understood when read in conjunction with the appended drawings. For the purpose of illustrating the invention, there are shown in the drawings exemplary and preferred embodiments of the invention; however, the disclosure is not limited to the specific methods, compositions, and devices disclosed. In addition, the drawings are not necessarily drawn to scale. In the drawings:

FIG. 1 depicts an illustrative blood glucose meter (left) and the structure of an exemplary test strip for use in the meter (right three panels). As shown in the upper right panel, carbon (or other conductive material) contacts may be disposed atop a substrate material. Insulation (middle right panel) may then be disposed to as to define a test region that includes at least some portion of the conductive contacts. Bioactive material (e.g. in ink or other form) may then be applied (lower right panel) so as to define the test region of the strip; in this illustrative figure, the test region is in electronic communication with the conductive contacts.

FIG. 2 provides sample nomenclature codes for the illustrative examples provided herein. FIG. 2 also provides thickness and surface finish information for the test samples.

FIG. 3 provides shrinkage data (in graphical and tabular form) for exemplary samples.

FIG. 4 provides Young's Modulus and % Strain at Yield data for exemplary (PPPBP/BPA PC; 1-3) and (PPPBP/BPA PC and PET; 4-5) samples.

FIG. 5 provides tear strength and transverse tear strength data for exemplary (PPPBP/BPA PC; 1-3) and (PPPBP/BPA PC and PET; 4-5) samples.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Any documents mentioned herein are incorporated herein in their entireties for any and all purposes.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. When referring to a value, the term “about” means the value and all other values within 10% of the value. For example, “about 10” means from 9 to 11 and all intermediate values, including 10.

In today's world, glucose strips are becoming progressively thinner and thinner, e.g., down to about 0.35 mm in some instances. Existing materials, however, do not always reliably meet performance requirements at these dimensions, and existing materials (e.g., those that include flame resistance additives) also pose certain regulatory challenges, including challenges in manufacture and disposal.

In one embodiment, the present disclosure provides, inter alia, new amorphous-crystalline alloy materials and articles (e.g., films) that simplify the manufacturing process and to increase the productivity yield not only on the customer side but also in the film extrusion process. The disclosed materials (PC/PBT, PC/PET, and others) are well-suited to film extrusion for their balance of mechanical performance and shrinkage performance at high temperature.

Because of their printability and stiffness at, e.g., 0.35 mm thickness, the disclosed PC/PBT films may be used in medical strips. As compared with certain PET films, the PC/PBT based films exhibit advantages in color, stiffness, shrinkage, and printability, among other characteristics. Exemplary films may include PBT, PC, mono zinc phosphate and hindered phenol stabilizers so as to extrude thin films having thicknesses in the range of from about 0.18 to about 0.45 mm, including films at a 0.35 mm thickness. The extruded films of this developed PC/PBT based resin have a very good appearance, mechanical properties and shrinkage performance at high temperature. SABIC's VALOX™, LEXAN™, XHT™, and ULTEM™ materials are all considered especially suitable base materials for the disclosed articles, although these materials are not exclusive. A strip according to the present disclosure may include one or more films (e.g., a PET film, a color film, and the like) that surmounts the strip.

The following aspects are illustrative only and do not limit the scope of the present disclosure. All aspects are combinable in any manner.

Aspect 1. A test element, comprising a substrate strip comprising a polymer blend having a glass transition temperature of greater than about 170 degrees C. as measured using a differential scanning calorimetry method, the substrate strip having a thickness in the range of from between about 0.10 mm and about 0.50 mm, at least one test field that comprises a bioactive detection material, at least one conductive portion supported by the substrate strip and in electronic communication with the detection material, at least one optical window supported by the substrate strip and in optical communication with the bioactive detection material, or both, and the test field being configured to receive a fluid sample.

By bioactive is meant a material that interacts with a biological material, e.g., a material that reacts with blood glucose, with saliva, and the like. The bioactive material may itself be biological in origin, but bioactive material may also be synthetic or even both biological and synthetic in origin. Enzymes are one exemplary bioactive material.

The glass transition temperature of the polymer blend may be, e.g., from about 160 degrees C. to about 250 degrees C., or from about 170 degrees C. to about 240 degrees C., or from about 180 degrees C. to about 230 degrees C., or from about 190 degrees C. to about 220 degrees C., or even from about 200 degrees C. to about 210 degrees C.

A test element may be, inter alia, configured as a glucose test strip for use in a glucose monitoring device, e.g., a home-use or hospital-use meter. The strip may be virtually any shape in cross-section, e.g., rectangular, square, circular, dog-bone, tapered, flared, and the like. The aspect ratio of the test element may be from 1:1 to about 100:1, or from 1:1 to about 50:1, or from 1:1 to about 20:1, or from 1:1 to about 10:1, or from 1:1 to about 5:1, or from 1:1 to about 2:1. The test element may also be configured as an insertable component (strip, stick, capillary, and the like) for applications other than glucose monitoring. For example, the element may be configured for use in urine testing, pregnancy testing, or for testing of bloodborne molecules. A test element may include one or more reagents (e.g., acid, base, preservative, PCR reagents, and the like) disposed thereon or even therein.

A test element may include a bar code, chip, a RFID device, or other indicia to demonstrate its origin, type, functionality, age, orientation during use, or any combination thereof. A reader device that interfaces with a testing element may include one or more features (e.g. bar code reader) that interacts with the test element (e.g., that checks the bar code on the test element to determine whether the bioactive material on the test element is in fact still active). Based on this interaction, the reader device may indicate to the user that the test element is suitable for use or may advise the user that the test element warrants replacement. A test element may also be shaped in a way such that it may only interface with a reader device in only one way. In this way, a test strip may be shaped such that it only inserts into a reader in the orientation that places the test field into proper orientation/position for the reader.

The thickness of the test strip may be from about 0.10 mm to about 0.50 mm, or from about 0.15 mm to about 0.40 mm, or even from about 0.20 mm to about 0.30 mm, including any and all intermediate values. Test strips having a thickness of about 0.35 mm are especially suitable.

The test field may be—as described elsewhere—a region that is sensitized to blood glucose or other biomarkers; sensitizing to blood glucose is considered especially suitable. The test field may include a variety of bioactive materials, as described elsewhere herein.

It should be understood that the test field may be in fluid communication with the environment exterior to the test element, or may be configured so that it may placed into fluid communication (e.g., by peeling-back of a protective film, by puncturing a protective barrier, or by sliding a cover so as to expose the test field) with the environment exterior to the test element. In this way, a test element may be maintained in ready condition and protected from the elements until the time of use.

Aspect 2. The test element of aspect 1, wherein the polymer comprises a polycarbonate (PC). Polycarbonates may include a PPPBP-BPA PC, which PC is considered especially suitable for the presently disclosed technology. Suitable such PCs are described in US2015/0232614 (incorporated herein in its entirety for any and all purposes) and include, e.g., a p-cumylphenol capped poly(65 mol % BPA carbonate)-co-(35 mol % 3,3-bis(4-hydroxyphenyl)-2-phenylisoindolin-1-one (PPPBP) carbonate) copolymer. PPPBP can also be referred to by the following names: 2-Phenyl-3,3-bis(4-hydroxyphenyl)phthalimidine; N-Phenyl-3,3-bis(4-hydroxyphenyl)phthalimidine; 3,3-bis(4-hydroxyphenyl)-2-phenylisoindolin-1-one; 3,3-bis(4-hydroxyphenyl)-2-phenyl-2,3-dihydro-1H-isoindol-1-one copolymer (e.g., MW=25000). PPPBP has, e.g., CAS #6607-41-6.

The term “PPPBP-PC” refers to a polycarbonate copolymer comprising repeating carbonate units derived from PPPBP and at least one other dihydroxy monomer such as a bisphenol A. For example, PPPBP-PC can be a polycarbonate copolymer comprising PPPBP and bisphenol A monomer units.

Other polycarbonates—in addition to PPPBP-BPA polycarbonate—are also considered suitable for the disclosed technology. Polycarbonate as used herein refers to an oligomer or a polymer comprising residues of one or more monomers, joined by carbonate linkages. Polycarbonate (and other polymers used herein) may be cross-linked, and may also feature end cap groups. Exemplary chain-stoppers include certain monophenolic compounds (i.e., phenyl compounds having a single free hydroxy group), monocarboxylic acid chlorides, monocarboxylic acids, and/or monochloroformates. Phenolic chain-stoppers are exemplified by phenol and C₁C₂₂ alkyl-substituted phenols such as p-cumyl-phenol, octylphenol, resorcinol monobenzoate, and p-tertiary-butylphenol, cresol, and monoethers of diphenols, such as p-methoxyphenol. Exemplary chain-stoppers also include cyanophenols, such as for example, 4-cyanophenol, 3-cyanophenol, 2-cyanophenol, and polycyanophenols. Alkyl-substituted phenols with branched chain alkyl substituents having 8 to 9 carbon atoms can be specifically be used.

Endgroups can be derived from the carbonyl source (i.e., the diaryl carbonate or carbonate precursor, or first linker moiety), from selection of monomer ratios, incomplete polymerization, chain scission, and the like, as well as any added endcapping groups, and can include derivatizable functional groups such as hydroxy groups, carboxylic acid groups, or the like. In an embodiment, the endgroup of a polycarbonate can comprise a structural unit derived from a diaryl carbonate, where the structural unit can be an endgroup. PCP is considered a suitable engroup. In a further embodiment, the endgroup is derived from an activated carbonate. Such endgroups can derive from the transesterification reaction of the alkyl ester of an appropriately substituted activated carbonate, with a hydroxy group at the end of a polycarbonate polymer chain, under conditions in which the hydroxy group reacts with the ester carbonyl from the activated carbonate, instead of with the carbonate carbonyl of the activated carbonate. In this way, structural units derived from ester containing compounds or substructures derived from the activated carbonate and present in the melt polymerization reaction can form ester endgroups. In an embodiment, the ester endgroup derived from a salicylic ester can be a residue of bis(methyl salicyl) carbonate (BMSC) or other substituted or unsubstituted bis(alkyl salicyl) carbonate such as bis(ethyl salicyl) carbonate, bis(propyl salicyl) carbonate, bis(phenyl salicyl) carbonate, bis(benzyl salicyl) carbonate, or the like. In a specific embodiment, where BMSC is used as the activated carbonyl source, the endgroup is derived from and is a residue of BMSC.

Polycarbonates may include one or more siloxane blocks. Suitable such compositions are described in United States published applications US2014/0234629 and 2014/0326162, both of which are incorporated herein by reference in their entireties for any and all purposes.

Aspect 3. The test element of aspect 2, wherein the polymer further comprises PET. As described elsewhere herein, the ratio of PET to PC may vary, e.g., from 100:1 to 1:100, or from 10:1 to 1:10, or from 5:1 to 1:5, or even from 2:1 to 1:2, e.g., 1:1.

Aspect 4. The test element of aspect 1, wherein the polymer comprises a PC-PBT blend. As described elsewhere herein, the ratio of PBT to PC may vary, e.g., from 100:1 to 1:100, or from 10:1 to 1:10, or from 5:1 to 1:5, or even from 2:1 to 1:2.

Aspect 5. A test element, comprising: a substrate strip comprising a PEI (polyetherimide) and having a glass transition temperature of greater than about 170 degrees C. as measured using a differential scanning calorimetry method, the substrate strip having a thickness in the range of from between about 0.10 mm and about 0.50 mm, at least one test field that comprises a bioactive detection material, at least one conductive portion supported by the substrate strip and in electronic communication with the detection material, at least one optical window supported by the substrate strip and in optical communication with the bioactive detection material, or both, and the test field being configured to receive a fluid sample.

A test strip may comprise PC-PET, PC-PBT, PEI, PC-PEI, PC-PEI-PBT, PC-PET-PEI, PC-PEI-PET-PBT, PC-PEI-PBT, or any combination thereof. PC-PET and PC-PBT are considered especially suitable combinations.

Polyetherimides may comprise polyetherimides homopolymers (e.g., polyetherimidesulfones) and polyetherimides copolymers. The polyetherimide can be selected from (i) polyetherimidehomopolymers, e.g., polyetherimides, (ii) polyetherimide co-polymers, and (iii) combinations thereof. Polyetherimides are known polymers and are sold by SABIC Innovative Plastics under the ULTEM®*, EXTEM®*, and Siltem* brands (Trademark of SABIC Innovative Plastics IP B.V.).

In an aspect, a polyetherimide can be of formula (1):

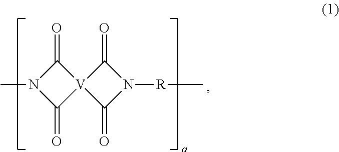

wherein a is more than 1, for example 10 to 1,000 or more, or more specifically 10 to 500.

The group V in formula (1) is a tetravalent linker containing an ether group (a “polyetherimide” as used herein) or a combination of an ether groups and arylenesulfone groups (a “polyetherimidesulfone”). Such linkers include but are not limited to: (a) substituted or unsubstituted, saturated, unsaturated or aromatic monocyclic and polycyclic groups having 5 to 50 carbon atoms, optionally substituted with ether groups, arylenesulfone groups, or a combination of ether groups and arylenesulfone groups; and (b) substituted or unsubstituted, linear or branched, saturated or unsaturated alkyl groups having 1 to 30 carbon atoms and optionally substituted with ether groups or a combination of ether groups, arylenesulfone groups, and arylenesulfone groups; or combinations comprising at least one of the foregoing. Suitable additional substitutions include, but are not limited to, ethers, amides, esters, and combinations comprising at least one of the foregoing.

The R group in formula (1) includes but is not limited to substituted or unsubstituted divalent organic groups such as: (a) aromatic hydrocarbon groups having 6 to 20 carbon atoms and halogenated derivatives thereof; (b) straight or branched chain alkylene groups having 2 to 20 carbon atoms; (c) cycloalkylene groups having 3 to 20 carbon atoms, or (d) divalent groups of formula (2):

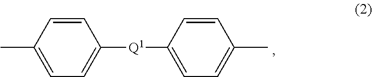

wherein Q1 includes but is not limited to a divalent moiety such as —O—, —S—, —C(O)—, —SO₂—, —SO—, —C_{y}H_{2y}— (y being an integer from 1 to 5), and halogenated derivatives thereof, including perfluoroalkylene groups.

In an embodiment, linkers V include but are not limited to tetravalent aromatic groups of formula (3):

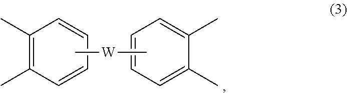

wherein W is a divalent moiety including —O—, —SO₂—, or a group of the formula —O—Z—O— wherein the divalent bonds of the —O— or the —O—Z—O— group are in the 3,3′, 3,4′, 4,3′, or the 4,4′ positions, and wherein Z includes, but is not limited, to divalent groups of formulas (4):

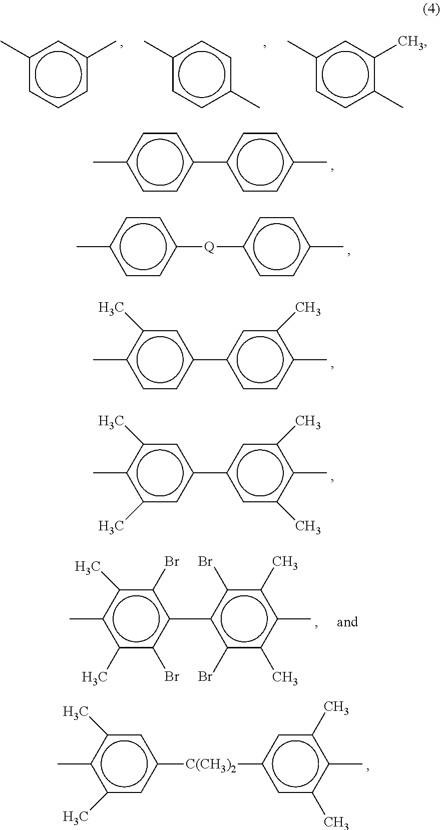

wherein Q includes, but is not limited to a divalent moiety including —O—, —S—, —C(O)—,

—SO₂—, —SO—, —C_{y}H_{2y}— (y being an integer from 1 to 5), and halogenated derivatives thereof, including perfluoroalkylene groups.

In an aspect, the polyetherimide comprise more than 1, specifically 10 to 1,000, or more specifically, 10 to 500 structural units, of formula (5):

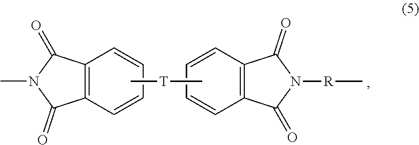

wherein T is —O— or a group of the formula —O—Z—O— wherein the divalent bonds of the —O— or the —O—Z—O— group are in the 3,3′, 3,4′, 4,3′, or the 4,4′ positions; Z is a divalent group of formula (3) as defined above; and R is a divalent group of formula (2) as defined above.

Aspect 6. The test element of any of aspects 1-5, wherein the bioactive material comprises a material reactive to blood glucose. Such materials include, without limitation, enzymes, polynucleotides, peptides, other proteins, and the like.

Aspect 7. The test element of aspect 6, wherein the bioactive material comprises glucose oxidase, glucosehydrogenase, or any combination thereof. Materials that undergo a detectable change (e.g., change in conformation) when contacted with blood glucose are considered especially suitable.

Aspect 8. The test element of any of aspects 1-7, wherein the substrate strip polymer blend is characterized as having a mold shrinkage in the machine direction (MD) in the range of less than about 4% (e.g., about 3%, about 2%, about 1%, or less) upon exposure to 170 deg. C. for 8 hours.

Aspect 9. The test element of any of aspects 1-7, wherein the substrate strip polymer blend is characterized as having a mold shrinkage in the transverse direction (TD) in the range of less than about 4% (e.g., about 3%, about 2%, about 1%, or less) upon exposure to 170 deg C. for 8 hours.

Aspect 10. The test element of any of aspects 1-9, wherein the substrate has a color dimension a of from about −2.0 to about −4.0, when measured at 1 mm Color, average.

Aspect 11. The test element of any of aspects 1-9, wherein the substrate has a color dimension b of about −7.0 to about −10.0, when measured at 1 mm Color, average.

Aspect 12. The test element of any of aspects 1-9, wherein the substrate has an L of from about 70 to about 85, when measured at 1 mm Color, average.

Without being bound to any particular theory, the disclosed test elements (or, at least their polymeric substrates) may be configured so that they are white in color. This facilitates locating the test elements and also facilitates optical operations (e.g., detection of color and other changes) related to the elements' operation.

Aspect 13. The test element of any of aspects 1-12, wherein the substrate polymer blend has a flexural modulus in the range of from about 1800 to about 2300 MPa, e.g., from 2000 to about 2200 MPa. Substrates with flexural moduli above about 2000 MPa (e.g., from about 2000 MPa to about 2500 MPa) are considered especially suitable.

Aspect 14. The test element of any of aspects 1-12, wherein the substrate polymer blend has a flexural stress at break of from about 75 to about 90 MPa, e.g., from about 80 to about 85 MPa. Substrates with flexural stresses at break about about 90 MPa (e.g., from about 90 MPa to about 120 MPa) are considered especially suitable.

Aspect 15. The test element of any of aspects 1-12, wherein the substrate polymer blend has a modulus of elasticity of from about 2200 to about 2600 MPa, e.g., from about 2300 to about 2400 MPa. Substrates with moduli of elasticity above about 2400 MPa (e.g., from about 2400 MPa to about 2600 MPa) are considered especially suitable.

Aspect 16. The test element of any of aspects 1-12, wherein the substrate polymer blend has a stress at break of from about 50 to about 60 MPa, e.g., from about 53 to about 57 MPa.

Aspect 17. The test element of any of aspects 1-12, wherein the substrate polymer blend has an elongation at break of from about 80 to about 180%, e.g., from about 100 to about 150%, or even from about 110 to about 130%. Substrates with elongations at break above about 120% (e.g., from about 120% to about 150%) are considered especially suitable.

Aspect 18. The test element of any of aspects 1-12, wherein the substrate polymer blend has a ductility of from about 50 to about 100%, e.g. from about 70 to about 85%. Substrates that have a HDT of about about 100 deg. C. (e.g., from about 100 deg. C. to about 150 deg. C.) are considered especially suitable.

Aspect 19. The test element of any of aspects 1-12, wherein the substrate polymer blend has an impact strength of from about 50 to about 1000 J/m, e.g. from about 100 to about 800 J/m, or from about 200 to about 500 J/m, or even from about 300 to about 400 J/m.

Aspect 20. The test element of aspect 4, wherein the ratio (wt %) of PC to PBT is from about 40:60 to about 99:1.

Aspect 21. The test element of aspect 20, wherein the ratio (wt %) of PC to PBT is from about 50:50 to about 90:10.

Aspect 22. The test element of aspect 21, wherein the ratio (wt %) of PC to PBT is from about 60:40 to about 70:30.

Aspect 23. The test element of aspect 3, wherein the ratio (wt %) of PC to PET is from about 60:40 to about 90:10.

Aspect 24. The test element of aspect 22, wherein the ratio (wt %) of PC to PET is from about 70:30 to about 80:20.

Aspect 25. The test element of any of aspects 1-24, wherein the test element is applied to a flexible transport tape. Suitable such transport tapes may be polymeric or even metallic in nature. The application may be facilitated by adhesive, magnetic forces, static electricity, and the like. A transport tape may be perforated or even wound on a spool.

Aspect 26. The test element of any of aspects 1-25, wherein the test element comprises a fibrous web contacting the test field so as to effect liquid sample spreading. The fibrous web may be textile in nature, and may include hydrophilic and/or hydrophobic materials. The web is suitably configured (in material, size and shape) to encourage fluid transport to the test field. The web may direct fluid from another part of the test element to the test field. The web may overlie at least some of the test field. In some embodiments, the web is configured to wick to the test field a fluid sample applied at some region of the test element.

Aspect 27. A system, comprising a test element according to any of claims 1-26, and a reader device being configured to receive the test element, the reader device also being configured to detect a change in signal of the test element related to an interaction between the bioactive detection material and subject blood. Exemplary reader devices include glucose test meters well-known to those of skill in the art. The changes in signal may be electrical (including chemical) or optical in nature.

Aspect 28. The system of aspect 27, wherein the signal comprises an electrical signal. The electrical signal may be detected by a voltmeter, an ammeter, and the like.

Aspect 29. The system of aspects 27 or 28, wherein the signal comprises an optical signal. The optical signal may be a change in color, a change in intensity, the appearance or disappearance of an optical signal, or any combination of the foregoing.

Aspect 30. A method of printing a plurality of test elements, comprising forming substrates from a supply of PC and PBT, PEI, PC and PET, or any combination thereof; disposing an amount of bioactive material reactive with blood glucose so as to form a test field supported by the substrate; disposing an amount of conductive material so as to form a conductive portion that is in electronic communication with the bioactive material and is supported by the substrate, disposing an amount of optically transmissive material into optical communication with the bioactive material, or both, so as to form the test element. The printing may be performed in batch, semi-batch, or continuous manner.

The formation may be effected by film deposition, extrusion, coating, or by other methods known to those of skill in the art. Suitable bioactive materials are described elsewhere herein, and the deposition of these materials may be effected by dripping, pipetting, spraying, lithography, or by other methods known to those skilled in the art.

Aspect 31. The method of aspect 30, further comprising cutting the substrates. This may be accomplished by saws, dicing, stamping, or by other methods known to those in the art for forming specific shapes from films and/or layers.

Aspect 32. The method of aspects 30 or 31, further comprising disposing the substrates on a flexible transport tape. This may be done at the time of substrate formation, e.g., forming the substrates atop the transport tape, or at some time following substrate formation.

Aspect 33. A test element, comprising: a substrate strip comprising a composition that comprises a polymer blend of an amorphous polymer having a Tg of greater than about 130 deg. C. and a crystalline polymer, the substrate strip having a thickness in the range of from between about 0.10 mm and about 0.50 mm. The composition suitably has a MVR value, determined under ASTM D 1238, of greater than 10.

As described elsewhere herein, polycarbonate and PEI (polyetherimide) are both considered suitable amorphous polymers. A variety of crystalline polymers are considered suitable, including, e.g., polyesters. PBT, PET, PCT (polycyclohexylenedimethylene terephthalate), and PTT (polytrimethylene terephthalate) are considered especially suitable crystalline polymers.

It should be understood that the disclosed materials may include one or more colorants. TiO₂ may be used, e.g., for enhancing the whiteness of the material. TiO₂ may be present at, e.g., from 0.1 to about 3 wt %. Other colorants—including dyes, inks, paints, and pigments—may be used to adjust the color of the material.

The composition of the test element may have an MVR (determined by ASTM D 1238) in the range of from about 5 to about 17, or from about 7 to about 15, or from about 5 to about 13, or even from about 7 to about 11. The MVR may be modulated by selection of polycarbonate of a particular molecular weight. The MVR may be greater than 10, greater than 11, greater than 12, greater than 13, or even greater than 14. The MVR may be about 11, about 12, about 13, about 14, about 15, about 16, about 17, or even about 18.

Aspect 34. The test element of aspect 33 or any other foregoing aspect, wherein the amorphous polymer comprises a polycarbonate. LEXAN™ and other polycarbonates familiar to those of skill in the art are all considered suitable. It should be understood that the amorphous polymer portion of the substrate composition may include one, two, or more amorphous polymers.

Aspect 35. The test element of any of aspects 33-34 or any other foregoing aspect, wherein the polycarbonate comprises a PPPBP-BPA polycarbonate.

Aspect 36. The test element of aspect 33 or any other foregoing aspect, wherein the amorphous polymer comprises PEI (polyetherimide). SABIC's ULTEM™ is considered an especially suitable PEI.

Aspect 37. The test element of any of aspects 33-36 or any other foregoing aspect, wherein the crystalline polymer comprises PET, PBT, PCT, PTT, or any combination thereof.

Aspect 38. The test element of any of aspects 33-37 or any other foregoing aspect, wherein the crystalline polymer comprises PET. The

Aspect 39. The test element of any of aspects 33-37 or any other foregoing aspect, wherein the crystalline polymer comprises PBT.

Aspect 40. The test element of any of aspects 33-37 or any other foregoing aspect, wherein the crystalline polymer comprises PCT.

Aspect 41. The test element of any of aspects 33-37 or any other foregoing aspect, wherein the crystalline polymer comprises PTT. It should be understood that the crystalline polymer portion of the substrate may comprise one, two, or more crystalline polymers.

Aspect 42. The test element of any of aspects 33-41 or any other foregoing aspect, wherein the crystalline polymer is present at from about 2 wt % to about 40 wt % of the composition of the substrate strip. The crystalline polymer may be present at, e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 wt %, as well as any intermediate value. The crystalline polymer may also be present at from about 40 wt % to about 50%, in some embodiments.

Aspect 43. The test element of any of aspects 33-42 or any other foregoing aspect, wherein the crystalline polymer is present at from about 20 wt % to about 30% of the composition of the substrate strip.

Aspect 44. The test element of any of aspects 33-43 or any other foregoing aspect, wherein the wt % ratio of amorphous polymer to crystalline polymer in the composition of the substrate strip is from about 10:1 to 1:10. The ratio may also be, e.g., about 9:1 to 1:9, about 8:1 to 1:8, about 7:1 to 1:7, about 6:1 to 1:6, about 5:1 to 1:5, about 4:1 to 1:4, about 3:1 to 1:3, about 2:1 to 1:2, or even about 1:1.

Aspect 45. The test element of any of aspects 33-44 or any other foregoing aspect, wherein the substrate strip has a tensile modulus of elasticity of between about 2300 MPa and about 2700 MPa, e.g., from 2400 to 2600 MPa, or even about 2500 MPa.

Aspect 46. The test element of any of aspects 33-45 or any other foregoing aspect, wherein the substrate strip has a tensile stress at break of from about 50 MPa to about 100 MPa, e.g., from about 60 to about 90 MPa, or from about 70 to about 80 MPa.

Aspect 47. The test element of any of aspects 33-46 or any other foregoing aspect, wherein the substrate strip has a tensile elongation at break of from about 110% to about 140%, e.g., from about 120% to about 130%, or about 120%.

Aspect 48. The test element of any of aspects 33-47 or any other foregoing aspect, wherein the test element comprises at least one test field that comprises a bioactive detection material. Suitable bioactive detection materials are described elsewhere herein, and include enzymes. Bioactive materials that are sensitive to blood glucose are considered especially suitable.

Aspect 49. The test element of any of aspects 33-48 or any other foregoing aspect, wherein the substrate strip is characterized as having a mold shrinkage in the machine direction (MD) in the range of less than about 4% upon exposure to 170 deg C. for 8 hours. Suitable mold shrinkages may be 4.0, 3.9, 3.8, 3.7, 3.6, 3.5, 3.4, 3.3, 3.2, 3.1, 3.0, 2.9, 2.8, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or even 0%. Negative shrinkage values are also suitable.

Aspect 50. The test element of any of aspects 33-49 or any other foregoing aspect, wherein the substrate strip is characterized as having a mold shrinkage in the transverse direction (TD) in the range of less than about 2% upon exposure to 170 deg C. for 8 hours. Suitable mold shrinkages may be 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, or even 0%. Negative shrinkage values are also suitable. It should be understood that a test element may have the aforementioned mold shrinkage in the machine direction (MD) characteristics, may have the aforementioned mold shrinkage in the transverse direction (TD) characteristics, or both.

Aspect 51. A system, comprising: a test element according to any of aspects 33-50 or any other foregoing aspect, and; a reader device being configured to receive the test element, the reader device also being configured to detect a change in signal of the test element related to an interaction between the test element and a biological sample. The reader may, as describe elsewhere herein, be configured to apply a reagent, a voltage, an illumination, or other stimulus to the test element. The test element may include a bioactive material that interacts with the biological sample (e.g., blood, urine, vomit, plasma, saliva, and other biological samples).

Aspect 52. A method, comprising: contacting a biological sample to a test element according to any of aspects 33-50 or any other foregoing aspect; and detecting a change in signal of the test element related to an interaction between the test element and the biological sample.

Aspect 53. A patient testing method, comprising, contacting a biological sample from a patient and a test element according to any of the foregoing aspects, and detecting a change in signal of the test element related to an interaction between the test element and the biological sample.

Aspect 54. The patient testing method of aspect 53, further comprising engaging the test element with a reader device configured to detect the change in signal. A reader device may be as described elsewhere herein. A reader and a test element may be configured to engage in a complementary fashion; e.g., the test element may be shaped so as to fit into the reader in only one orientation. In this way, the engagement between the test element and the reader may place a bioactive material of the test element into proper position for interrogation, illumination, or other processing by the reader.

Aspect 55. The patient testing method of aspects 53 or 54, further comprising assigning a treatment protocol to the patient. Treatment protocols include, e.g., administration of insulin, administration of a sugar, administration of an antibiotic, administration of a hormone, administration of a blood pressure lowering agent, and the like. The reader may be configured to assign the treatment protocol—as one example, a reader may be configured to assign insulin administration to a user whose blood glucose level is above a certain level.

Aspect 56. A method of fabricating a test element, comprising: from a film that comprises a polymer blend of an amorphous polymer having a Tg of greater than about 130 deg. C. and a crystalline polymer, the film having a thickness in the range of from between about 0.10 mm and about 0.50 mm, and the film having a tensile modulus of elasticity of between about 2300 MPa and about 2700 MPa, cutting the film to one or more predetermined shapes.

Aspect 57. The method of aspect 56, further comprising disposing a bioactive material such that the bioactive material is supported by the film. The film may also have one or more properties according to any of aspects 33-50, e.g., an MVR of greater than 10.

Additional Disclosure

The following disclosure provides exemplary, non-limiting technical results from testing of illustrative embodiments of the disclosed technology. These results are illustrative only and do not serve to limit the scope of the present invention.

The following table provides a list of the standards and testing conditions used herein.

| | Standards | Testing Conditions |
|---|---|---|
| Melt Volume Rate (MVR)*, Melt | ASTM D 1238 | 250 deg. C., 2.16 |
| Mass-flow Rate (MFR)* | | Kg |
| *PC MVR may also be measured | | |
| at 300 deg. C., 1.2 kg. | | |
| Uniaxial Tensile test | ASTM D 638 | 50 mm/min |
| Notched Izod Impact (NII) | ASTM D 256 | 5 lbf, 23° C., 3.2 |
| | | mm |
| Vicat softening temperature (VST) | ASTM D 1525 | 50N, 120° C./h |
| Shrinkage | (from outside | 170 C. 8 hrs |
| | source) | |
| HDT | ASTM D648 | 1.82 MPa, 6.4 mm, |
| | | unannealed |
| Flexural | ASTM D790 | 1.3 mm/min, 50 mm |
| | | span |
| Color | | |

Compounding, Molding

Typical compounding and molding procedures are described as follows:

Polycarbonate powders, which may contain different ratio of low-flow PC (LF PC, MFR: ˜3.5 g/10 mins), normal-flow PC(NF PC, MFR: ˜7 g/10 mins) and high-flow PC (HF PC, MFR: ˜27 g/10 mins), are pre-blended with the PBT, impactor, and necessary thermal stabilizer and other additive at certain loadings. The pre-blended PC/PBT were extruded using a twin extruder. The extruded pellets were molded in different shapes for mechanical tests.

Film Extrusion

Films at 8 mil-15 mil were extruded on a film lab line.

Exemplary Results

Table 1 shows the first formulation screen based on a PC/PBT blend, which contains Br PC and antimony oxide. As shown, formulations #2 and #4 shows favorable shrinkage at both directions. Samples 2 and 4 alter the ratio of PC and PBT, and a blend of about 30% PBT showed particularly favorable performance. Films at 350 micrometers were extruded in lab and sent to an evaluator for consideration, and the results of that consideration showed that sample 3 was also favorable in performance. Color matching may be accomplished by adding TiO2 to achieve a desired L-a-b value—as one example, 2.5 phr TiO2 was added in a formulation to achieve a desired L value.

TABLE 1

| | | Component | Unit | #1 | #2 | #3 | #4 |
|---|---|---|---|---|---|---|---|
| | | PCP | % | 58.4 | | | |
| | | Polycarbonatewith | % | | 68.5 | 43 | 60 |
| | | PCP end cap | | | | | |
| | | PBT | % | 30.62 | 30.62 | 37.44 | 37.14 |
| | | TBBPA/BPA COPOLYMER | % | | | | |
| | | WITH PCP ENDCAP | | | | | |
| Properties | MVR | 250 C., 2.16 kg, 360 s | cm³/10 min | 3.3 | 2 | 2.6 | 3.2 |
| | HDT | Deflection temp | ° C. | 101 | 103 | 89 | 100 |
| | Shrinkage | MD at 170 C., 8 hrs | % | −3.00% | −2.00% | −4.00% | −2.00% |
| | | TD at 170 C., 8 hrs | % | −1.00% | −1.00% | −1.00% | −1.00% |
| | Flex | Modulus | MPa | 2250 | 2140 | 2020 | 2050 |
| | | Stress at Break | MPa | 90 | 94 | 75 | 87 |
| | Tensile | Modulus of Elasticity | MPa | 2306 | 2361 | 2017 | 2269 |
| | | Stress at Break | MPa | 59 | 55 | 41 | 56 |
| | | Elongation at Break | % | 97 | 75 | 45 | 97 |
| | Nil | Ductility | % | 100 | 0 | 100 | 100 |
| | | Impact Strength | J/m | 879 | 141 | 800 | 755 |
| | Color @ 2 mm | L | — | 80.7 | 71.1 | 95.6 | 78.7 |
| | | a | — | −2.7 | −3.6 | −0.3 | −3 |
| | | b | — | −6 | −9.9 | 2.8 | −4.9 |
| | Color @ 1 mm | L | — | 73.5 | 61.2 | 94.7 | 71.5 |
| | | a | — | −2.4 | −3.3 | −0.6 | −3.3 |
| | | b | — | −7.8 | −13.1 | 1.6 | −8.6 |
| Application | Oven heating | 170 C., 8 hrs | — | NG | NG | NG | Pass |
| requirement | Stiffness | (bend by hand) | — | NG | NG | NG | NG |
| | check | | | | | | |
| | Die-cut check | check cracking line | — | Pass | Pass | Pass | Pass |
| | | after die cut | | | | | |
| | film color | visual inspection | — | Pass | Pass | Pass | Pass |

| | | Component | #5 | #6 | #7 | #8 |
|---|---|---|---|---|---|---|
| | | PCP | 15 | | | |
| | | Polycarbonatewith | | 63.3 | 69.74 | 49.74 |
| | | PCP end cap | | | | |
| | | PBT | 36.44 | 36.44 | 30 | 50 |
| | | TBBPA/BPA COPOLYMER | 29 | | | |
| | | WITH PCP ENDCAP | | | | |
| Properties | MVR | 250 C., 2.16 kg, 360 s | 7.5 | 8.3 | 7.1 | 13.1 |
| | HDT | Deflection temp | 93 | 98 | 102 | 88 |
| | Shrinkage | MD at 170 C., 8 hrs | −2.07% | −2.50% | −2.74% | −2.74% |
| | | TD at 170 C., 8 hrs | −1.50% | −1.87% | −2.29% | −2.35% |
| | Flex | Modulus | 1720 | 2230 | 2050 | 2190 |
| | | Stress at Break | 72 | 93 | 94 | 89 |
| | Tensile | Modulus of Elasticity | 2046 | 2448 | 2437 | 2460 |
| | | Stress at Break | 41 | 67 | 71 | 60 |
| | | Elongation at Break | 62 | 127 | 138 | 127 |
| | Nil | Ductility | 100 | 0 | 0 | 0 |
| | | Impact Strength | 603 | 118 | 134 | 89 |
| | Color @ 2 mm | L | 95 | 74.3 | 68.2 | 80.6 |
| | | a | −0.3 | −2.3 | −2.8 | −2 |
| | | b | 3.8 | −3.1 | −5.8 | −1.2 |
| | Color @ 1 mm | L | 94.3 | 62.3 | 55.1 | 73.9 |
| | | a | −0.7 | −3.5 | −2.6 | −2.6 |
| | | b | 2.6 | −9.5 | −11.6 | −5.6 |
| Application | Oven heating | 170 C., 8 hrs | Pass | Pass | Pass | Pass |
| requirement | Stiffness | (bend by hand) | NG | Pass | Pass | Pass |
| | check | | | | | |
| | Die-cut check | check cracking line | Pass | Pass | Pass | Pass |
| | | after die cut | | | | |
| | film color | visual inspection | Pass | Pass | Pass | Pass |

As shown above, samples 6, 7, and 8 exhibited comparatively positive performance in the application requirements of oven heating resistance, hand stiffness check, die-cut checking, and film color via visual inspection.

The above-listed examples are not limiting, and are illustrative only. Compositions according to the present disclosure may also include (but do not require) any one or more of a quencher, a flame retardant (FR) additive, a UV resistance additive, a stabilizer, or an impact modifier. The disclosed compositions may suitably exhibit the following properties (for a sample having a thickness of from about 0.10 mm to about 0.50 mm, e.g., for a sample having a thickness of 0.35 mm). This list is non-limiting, and any ranges provided herein are illustrative and non-limiting:

MVR: Suitably above 10, e.g., from 11 to 17, or even about 15.

HDT: Suitably above about 100 deg C.

Modulus (flex): Suitably above about 1900 MPa, e.g., from 2000 MPa to about 3000 MPa.

Stress at Break (flex): Suitably above about 80 MPa e.g., from 90 to about 130 MPa.

Modulus of Elasticity (tensile): Suitably above about 2300 MPa, e.g., from 2350 MPa to about 2700 MPa.

Stress at Break (tensile): Suitably above about 50 MPa, e.g., from 60 MPa to about 100 MPa.

Elongation at Break (tensile): Suitably above about 110%, e.g., from 110% to about 140%.

Table 2 below provides further performance data for illustrative alternative formulations that include, variously, polycarbonates, PBT, and PET. The table also provides performance data for these illustrative formulations.

TABLE 2

| | Item Description | Unit | Sample A | Sample B | Sample C | Sample D |
|---|---|---|---|---|---|---|
| 1 | PC (Mw = 30000) | % | | 32.57 | 31.82 | 11.57 |
| 2 | SAN encapsulated PTFE | % | | 0.75 | 1.5 | 1.5 |
| 3 | PET | % | 26.5 | 26.5 | | |
| 6 | Impact Modifier | % | 7 | 7 | 7 | 7 |
| 9 | PPPBP/BPA | % | 30 | 30 | 20 | 40 |
| | copolycarbonate | | | | | |
| 10 | Stabilized PC pellets | % | 33.33 | | 0.01 | 0.01 |
| 11 | PBT | % | | 0.01 | 39.46 | 39.71 |
| HDT | Deflection temp | | | | | |
| Shrinkage | MD at 160 C., 40 s | | 0.41 | 0.48 | | |
| | TD at 160 C., 40 s | | 0.14 | 0.11 | | |
| Tensile | Youngs modulus | Mpa | 2253.6 | 2064.4 | | |
| | Stress @Break | Mpa | 51.23 | 52.552 | | |
| | % Strain at break | % | 21.9 | 5.4 | | |
| | Stress @Yield | Mpa | 58.9 | 55.9 | | |
| | % Strain at Yield | % | 4.6 | 4.65 | | |
| | Tear strength | N/mm | 13.7 | 13.3 | | |

Table 3 below provides additional illustrative formulations that include PET and various polycarbonates. The table also provides performance data for these materials

TABLE 3

| | | Unit | Sample 05 | Sample 06 | Sample 07 | Sample 08 | Sample 09 | Sample 10 | Sample 11 |
|---|---|---|---|---|---|---|---|---|---|
| 1 | PET | % | 26.5 | 26.5 | 14 | 5 | 14 | 5 | 26.5 |
| 2 | PPPBP/BPA | % | 30 | 36.25 | 43.04 | 48.04 | 45.04 | 49.04 | 38.14 |
| | copolycarbonate | | | | | | | | |
| 3 | Stabilized | % | 33.08 | 36.25 | 39.5 | 43.5 | 40.5 | 45.5 | 38.14 |
| | polycarbonate | | | | | | | | |
| Shrinkage | MD at 160 C., 40 s | % | 0.02 | 0.02 | 0.02 | −0.02 | −0.01 | −0.01 | |
| Shrinkage | TD at 160 C., 40 s | % | −0.05 | −0.04 | −0.04 | −0.01 | −0.01 | −0.01 | |
| Tensile | Young's Modulus | MPa | 2423.6 | 3092 | 2874 | 2789 | 2476 | 2423 | |
| Tensile | Stress @ Break | MPa | 58.18 | 71.08 | 71.89 | 60.64 | 71.43 | 72.7 | |
| Tensile | % Strain at Break | % | 5.02 | 5.16 | 5.01 | 11.84 | 4.63 | 5.22 | |
| Tensile | Stress @ Yield | MPa | 59.44 | 71.01 | na | 72.69 | na | 71.55 | |
| Tensile | % Strain at Yield | % | 4.45 | 5.27 | na | 5.812 | na | 4.805 | |
| Tensile | Tear Strength | N/mm | 14 | 9.26 | 9.776 | 11.275 | 9.292 | 8.477 | |

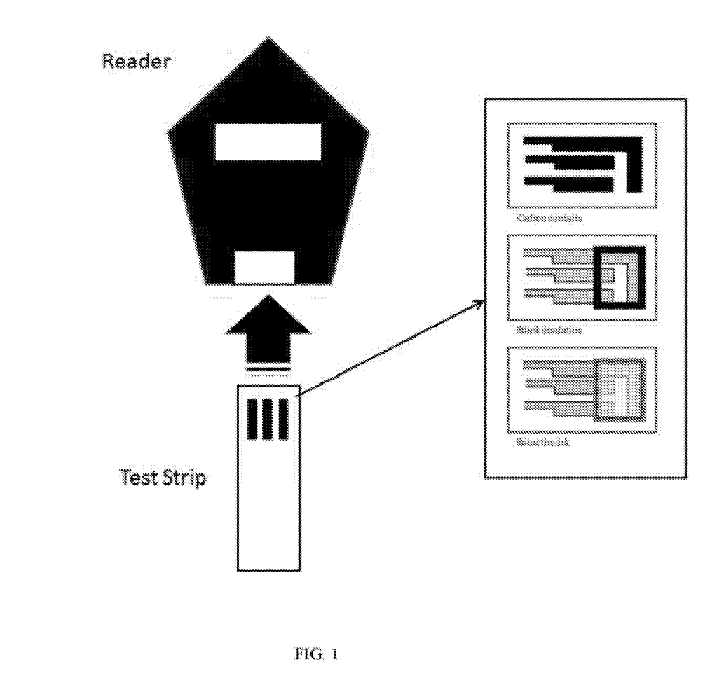

Additional description of the FIGs. is provided here. As explained above, FIG. 1 depicts an illustrative blood glucose meter (left) and the structure of an exemplary test strip for use in the meter (right three panels). The test strip may be contacted with a biological sample and then inserted (arrow) into the reader for processing. The reader may—as described elsewhere herein—introduce a reagent, illumination, an electrical current/voltage, or other stimulus to the test strip. The reader may then detect one or more signals related to an interaction of the test strip (or some component of the test strip) with the biological sample.

It should be understood that a user may contact the test strip with a biological sample before inserting the test strip into the reader. In some embodiments, the test strip is contacted with the biological sample before insertion into the reader.

As shown in the upper right panel, carbon (or other conductive material) contacts may be disposed (e.g., via deposition, printing, and other methods) atop a substrate material. Insulation (middle right panel) may then be disposed to as to define a test region that includes at least some portion of the conductive contacts. Bioactive material (e.g. in ink or other form) may then be applied (lower right panel) so as to define the test region of the strip; in this illustrative figure, the test region is in electronic communication with the conductive contacts.

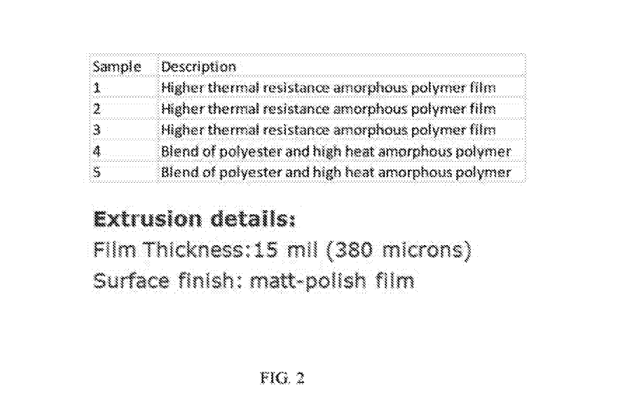

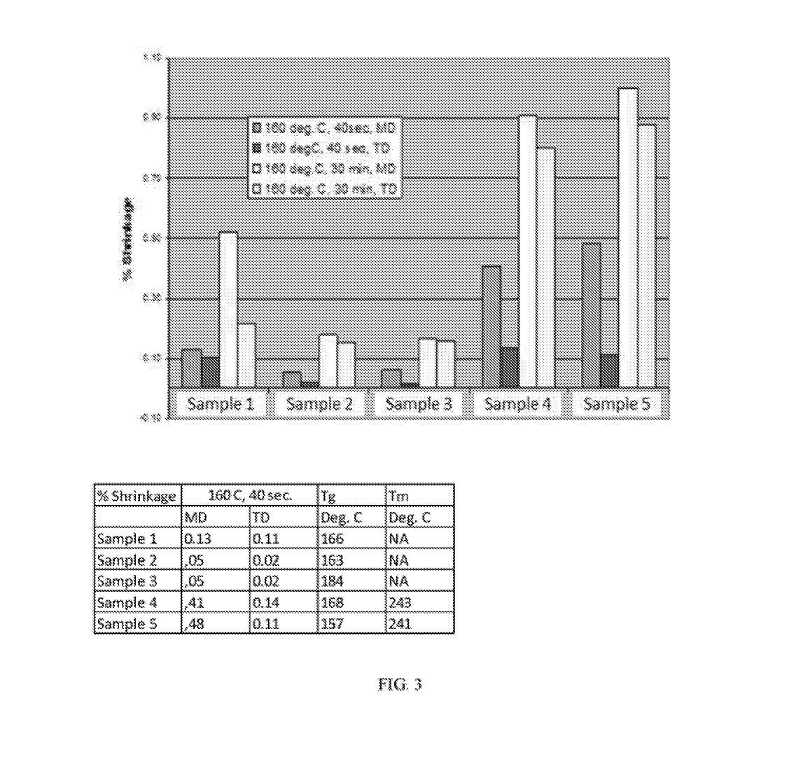

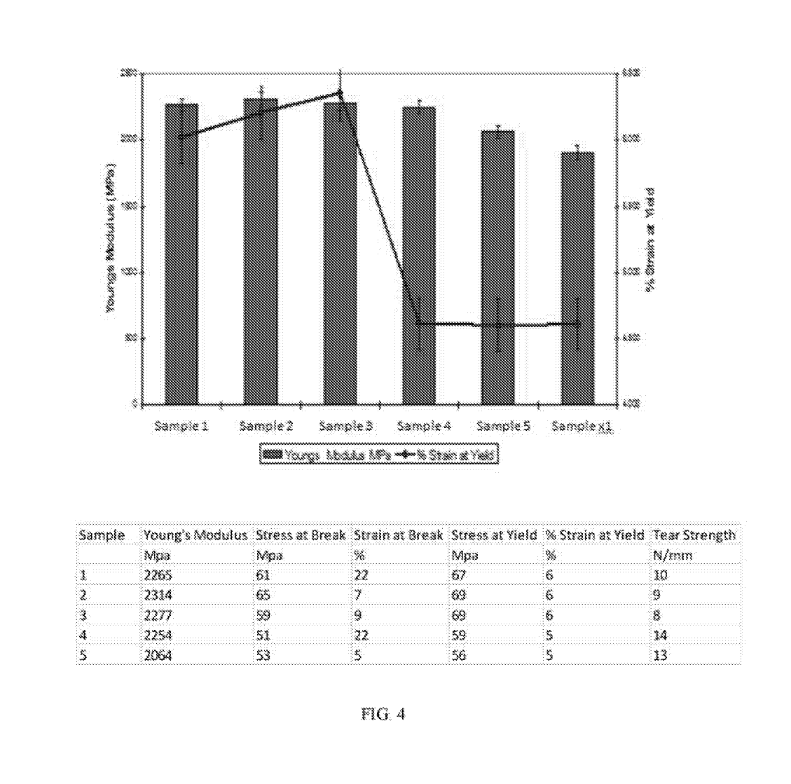

FIG. 2 provides sample nomenclature codes for the illustrative examples shown in FIG. 3 and FIG. 4. FIG. 2 also provides sample preparation information for the test samples, including thickness and surface finish.

FIG. 3 provides shrinkage data (in graphical and tabular form) for exemplary samples; samples 1-3 comprise a PPPBP-BPA PC with various other additives, sample 4 comprises PET blended with PPPBP-BPA PC and another PC; and sample 5 comprises comprise PET blended with PPPBP-BPA PC. As shown in the figure, the PPPBP-BPA PC—PET samples exhibited improved shrinkage performance (at various testing conditions) as compared to samples without included PET.

FIG. 4 provides Young's Modulus and % Strain at Yield (and other mechanical property) data for exemplary (PPPBP/BPA PC) and (PPPBP/BPA PC and PET) samples. As shown, the different material samples differed more in strain % at yield than they did in Young's modulus. (Sample X1 is a comparative sample of an existing PBT-containing product.)

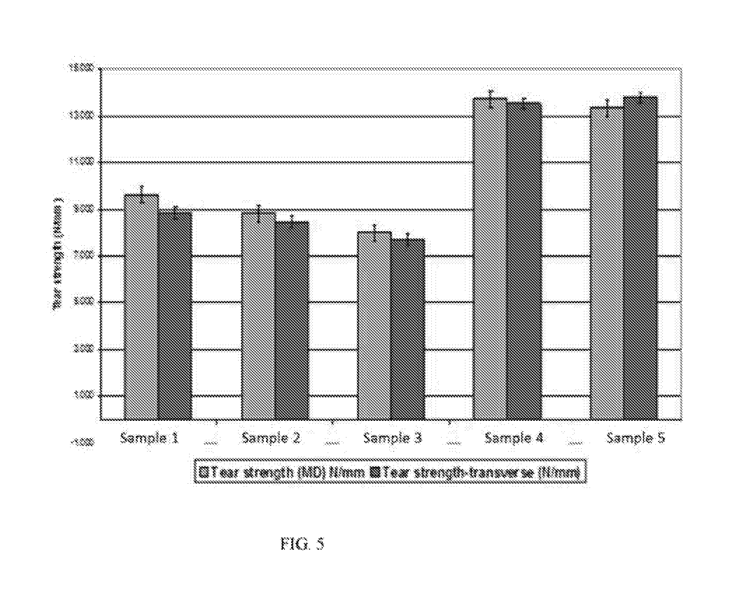

FIG. 5 provides tear strength and transverse tear strength data for exemplary (PPPBP/BPA PC; 1-3) and ET (PPPBP/BPA PC and PET; 4-5) samples. As FIG. 5 shows, the PC-PET blend samples exhibited improved tear strength—MD and TD—relative to the PC samples. Without being bound to any particular theory, Table 3 suggests that an increased PET content may correlate at least somewhat with increased tear strength.

What is claimed is:

1. A test element, comprising:
a substrate strip comprising a composition that comprises a polymer blend of an amorphous polymer having a Tg of greater than about 130 deg. C. and a crystalline polymer,
the substrate strip having a thickness in the range of from between about 0.10 mm and about 0.50 mm, and
the composition having a MVR, determined under ASTM D 1238, of greater than 10.

2. The test element of claim 1, wherein the amorphous polymer comprises a polycarbonate.

3. The test element of claim 1, wherein the polycarbonate comprises a PPPBP-BPA polycarbonate.

4. The test element of claim 1, wherein the amorphous polymer comprises PEI.

5. The test element of claim 1, wherein the crystalline polymer comprises PET, PBT, PCT, PTT, or any combination thereof.

6. The test element of claim 1, wherein the crystalline polymer comprises PET.

7. The test element of claim 1, wherein the crystalline polymer comprises PBT.

8. The test element of claim 1, wherein the crystalline polymer comprises PCT.

9. The test element of claim 1, wherein the crystalline polymer comprises PTT.

10. The test element of claim 1, wherein the crystalline polymer is present at from about 2 wt % to about 40 wt % of the composition of the substrate strip.

11. The test element of claim 1, wherein the composition has an MVR, determined under ASTM D 1238, of about 15.

12. The test element of claim 1, wherein the wt % ratio of amorphous polymer to crystalline polymer in the composition of the substrate strip is from about 10:1 to 1:10.

13. The test element of claim 1, wherein the substrate strip has a tensile modulus of elasticity of between about 2300 MPa and about 2700 MPa.

14. The test element of claim 1, wherein the substrate strip has a tensile stress at break of from about 50 MPa to about 100 MPa.

15. The test element of claim 1, wherein the substrate strip has a tensile elongation at break of from about 110% to about 140%.

16. The test element of claim 1, wherein (a) the substrate strip is characterized as having a mold shrinkage in the machine direction (MD) in the range of less than about 4% upon exposure to 170 deg C. for 8 hours, (b) wherein the substrate strip is characterized as having a mold shrinkage in the transverse direction (TD) in the range of less than about 2% upon exposure to 170 deg C. for 8 hours, or both (a) and (b).

17. A patient medical testing system, comprising:
a test element according to claim 1, and;
a reader device being configured to receive the test element, the reader device also being configured to detect a change in signal of the test element related to an interaction between the test element and a biological sample.

18. A patient testing method, comprising:
contacting a biological sample from a patient and a test element according to claim 1; and
detecting a change in signal of the test element related to an interaction between the test element and the biological sample.

19. The patient testing method of claim 18, further comprising engaging the test element with a reader device configured to detect the change in signal.

20. The patient testing method of claim 18, further comprising assigning a treatment protocol to the patient.
